(19) United States
(12) Patent Application Publication
(10) Pub. No.: US 2022/0114605 A1
(43) Pub. Date: Apr. 14, 2022

(54) INTERACTIVE QUESTIONNAIRE SYSTEM AND METHOD

(71) Applicant: Felix Mendelson, Los Gatos, CA (US)

(72) Inventor: Felix Mendelson, Los Gatos, CA (US)

(21) Appl. No.: 17/067,428

(22) Filed: Oct. 9, 2020

(51) Int. Cl. G06Q 30/02; G06Q 50/16; G06Q 50/14; G16H 10/20; G16H 50/20; G06F 16/955

(52) U.S. Cl. CPC G06Q 30/0203; G06Q 50/16; G06F 16/955; G16H 10/20; G16H 50/20; G06Q 50/14

(57) ABSTRACT

A method and system for providing interactive questionnaires is presented. The questionnaire includes a number of prompts for personal information together with compelling information that is presented during the questionnaire. The compelling information may be predetermined and/or be based on personal information provided from previous questions. The compelling information is related to the purpose of the questionnaire, such as selling real estate, selling merchandise, arranging for the providing of services, or diagnosing a medical condition, and is intended to provide the user with information that engages the person answering the questionnaire.

### BACKGROUND OF THE INVENTION

### Field of the Invention

The present invention generally relates to questionnaires, and more particularly to a method and system for providing an interactive questionnaire.

### Discussion of the Background

Websites often require users to provide personal information to gain access to services. Users are often wary of providing such information if they do not trust the website. This lack of trust can prevent users from using such websites, or from providing accurate information to the website. Thus, for example, a user may be presented with questions that they do not feel worth their time to answer or comfortable answering and they may navigate away from the web site.

An example of websites that requests personal information are those used for marketing real estate services. Such websites may, for example, require the user to provide their contact information and details of the property they wish to sell. This information may include, but is not limited to, the user's phone number or financial details, and specifics about the property to be sold, including the property address and other details. A user may not feel comfortable providing their name, email address, phone number, or property address without trusting the website, and may navigate away or provide false information.

Thus, there is a need in the art for a questionnaire that builds trust with a person responding to the questionnaire. There is also a need in the art for a questionnaire that encourages users to provide accurate personal information. Such a questionnaire should thus reduce the likelihood of users prematurely navigating away from the website before answering the questions posed by the questionnaire, providing their personal information, and/or completing a registration process, e.g. by opening an account. Such a questionnaire should be easy to incorporate into websites, be easy to use, and solicit honest answers and accurate information from users of the website.

### BRIEF SUMMARY OF THE INVENTION

The present invention overcomes disadvantages of prior art websites that request personal information by establishing trust with the user during the collection of the personal information. One embodiment provides an interactive process that, while prompting the user for personal information, also provides them with new and useful, or “compelling information.”

The term “personal information” is used herein, without limitation, to denote information provided by the user in response to questions prompted for by the website. The terms “compelling information,” “item of compelling information,” or “items of compelling information” are used herein, without limitation, to denote information the user may find engaging, relevant or useful, that is generated by the website and provided by the website to the user with the intention of inducing the user to continue to accurately answer all prompts for personal information. This inducement may, for example, occur by appealing to the users' curiosity and reciprocity and/or building trust with the website.

The present invention also overcomes the disadvantages of prior art websites for gathering personal information by prompting for personal information while also providing compelling information. In certain embodiments, the compelling information is relevant to the field of the website, and therefore the user's reason for using the website. The compelling information may become more personalized as the user progresses through the website, by utilizing user answers to questions previously asked within the website. Yet other embodiments prompt the user with the option of not having their personal information used to sell or market to them.

One embodiment provides a method of providing an on-line questionnaire for gathering personal information from a user. The method includes: providing a plurality of prompts for the user's personal information, where the plurality of prompts have a sequence including a last prompt; accepting a response from the user for each prompt of the plurality of prompts; determining one or more items of compelling information from one or more accepted responses; and providing the one or more items of determined compelling information to the user, where at least one of the one or more items of determined compelling information is provided to the user before the step of accepting the response to the last prompt. The method is such that the user is presented with compelling information while responding to the on-line questionnaire.

Another embodiment provides a system for providing an on-line questionnaire to gather personal information from a user. The system includes a server programmed to: provide a plurality of prompts for the user's personal information, where the plurality of prompts have a sequence including a last prompt; accept a response from the user for each prompt of the plurality of prompts; determine one or more items of compelling information from one or more accepted responses; and provide the one or more items of determined compelling information to the user, where at least one of the one or more items of determined compelling information is provided to the user before the response to the last prompt is accepted. The system is programmed such that the user is presented with compelling information while responding to the on-line questionnaire.

Yet another embodiment provides a method of providing an on-line questionnaire for personal information from a user. The method includes: providing one or more webpages to the user, where each webpage of the one or more webpages includes one or more prompts of a plurality of prompts, and where the plurality of prompts have a sequence including a last prompt; providing regions for responding to the prompts on the one or more webpages; accepting responses to the prompts from the regions; determining one or more items of compelling information from one or more accepted responses; and providing the determined one or more items of compelling information to the user on a webpage of the one or more webpages before accepting the response to the last prompt. The method is such that the user is presented with compelling information while responding to the on-line questionnaire.

A further embodiment provides a system for providing an on-line questionnaire for gathering personal information from a user. The system includes a server programmed to: provide one or more webpages to the user, where each webpage of the two or more webpages includes one or more prompts of a plurality of prompts, and where the plurality of prompts have a sequence including a last prompt; provide webpages including regions for responding to the prompts; accept responses to the prompts from the regions; determine one or more items of compelling information from one or more accepted responses; and provide the determined compelling information to the user before accepting the response to the last prompt. The system is programmed such that the user is presented with compelling information while responding to the on-line questionnaire.

These features together with the various ancillary provisions and features which will become apparent to those skilled in the art from the following detailed description, are attained by the questionnaire apparatus and method of the present invention, preferred embodiments thereof being shown with reference to the accompanying drawings, by way of example only, wherein:

### BRIEF DESCRIPTION OF THE SEVERAL VIEWS OF THE DRAWING

FIGS. 1 and 2 are schematic diagrams illustrative of one embodiment of a system for providing a multi-step questionnaire;

FIG. 3 is an embodiment of a screenshot of a first webpage of the system and is used to obtain a ZIP code for real estate the user intends to sell with the help of a real estate agent to be found on the website;

FIG. 4 is an embodiment of a screenshot of a second webpage of the system and is used to obtain the relationship of the user to the real estate being sold;

FIG. 5 is an embodiment of a screenshot of a third webpage of the system and is used to obtain personal information on the type of real estate being sold;

FIG. 6 is an embodiment of a screenshot of a fourth webpage of the system and is used to obtain more detailed personal information on the real estate being sold;

FIG. 7 is an embodiment of a screenshot of a fifth webpage of the system and is used to obtain personal information related to the selling of the real estate;

FIG. 8 is an embodiment of a screenshot of a sixth webpage of the system and is used to obtain more detailed personal information on the real estate being sold, including the user's estimate of the value of the real estate;

FIG. 9 is an embodiment of a screenshot of a seventh webpage of the system and is used to obtain personal information on the size of the improvements of the real estate being sold;

FIG. 10 is an embodiment of a screenshot of an eighth webpage of the system and is used to solicit photographs of the real estate being sold;

FIG. 11 is an embodiment of a screenshot of a ninth webpage of the system and is used to obtain the user's name and phone number and the address of the real estate being sold;

FIG. 12 is an embodiment of a screenshot of a tenth webpage of the system and is used to provide the user with an opportunity to review and edit the provided personal information and to send the user a verification code to the user's mobile telephone; and

FIG. 13 is an embodiment of a screenshot of an eleventh webpage of the system and is used to input the verification code and to set a password for an account on the website.

Reference symbols are used in the Figures to indicate certain components, aspects or features shown therein, with reference symbols common to more than one Figure indicating like components, aspects or features shown therein.

### DETAILED DESCRIPTION OF THE INVENTION

The present invention is a web-based system, and a method, that provides compelling information during an on-line questionnaire to motivate a website's user to provide accurate personal information to a website. In certain embodiments, the system and method provides the user one or more webpages forming a multi-step questionnaire requesting one or more piece of personal information. Some or all of the webpages also provide compelling information to the user based on the user's personal information provided earlier to the questionnaire, and further prompt the user to supply further personal information to the website. In certain embodiments, building the user's trust is furthered by including a notice with a promise that the user's personal information will not be used to sell or market to the user.

Compelling information may be generally classified as providing answers to questions that users have, or provide answers to questions that they have not yet considered, within the subject matter of the website. Compelling information can be dynamic or static. Static compelling information does not utilize the answers provided by the user while the user takes the questionnaire to form, determine, or select the compelling information. Dynamic compelling information utilizes the answers provided while the user takes the questionnaire to form, determine, or select the compelling information. Examples of dynamic compelling information may include, for example and without limitation, information deemed to be useful based on answers provided previously in the questionnaire.

In certain embodiments, compelling information becomes more specific as more personal information is gathered. In yet another embodiment the system provides compelling information that is relevant to the field of the website and/or the user's specific situation, problem, or need. The nature and amount of detail provided in the compelling information depends on the website and the users, and in general requires an expert in the field of the website to identify what compelling information to deliver to users, what information the target user would find valuable.

In other embodiments, users navigate from one webpage to the next by use of a button labeled “more information,” wherever more compelling information will be provided. This message encourages users to not navigate away by implying that the user will receive more information, and simultaneously builds trust by forewarning that the user may be asked for more personal information on the following page, or both. In other embodiments, the compelling information may provide guidance to the user.

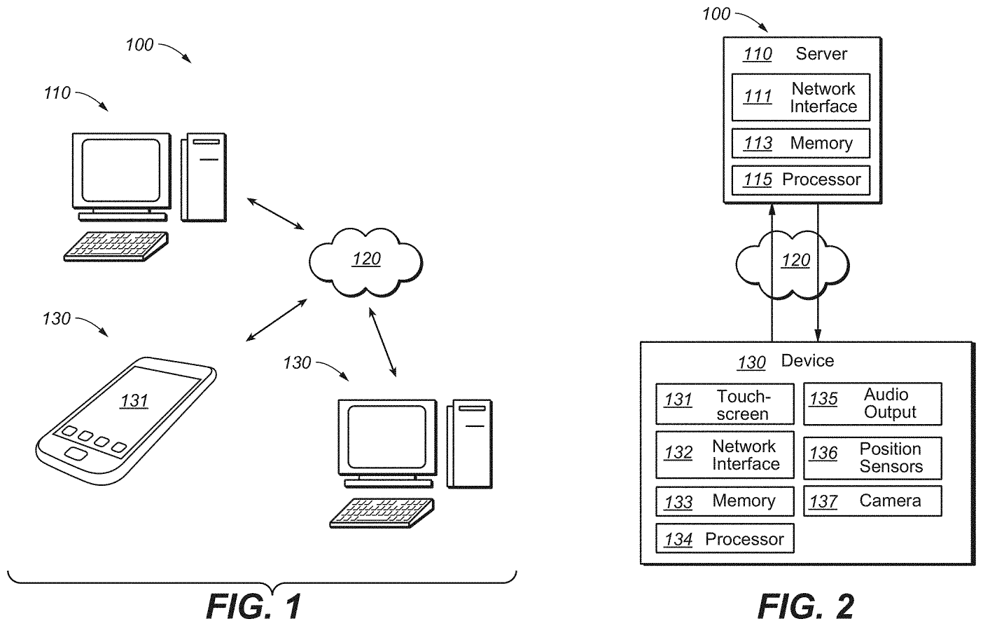

By way of example, the following discussion of the various embodiments are provided, without limitation, with reference to the schematic diagrams of FIGS. 1 and 2, for a system 100 that presents a website for matching real estate agents with home sellers or, alternatively for facilitating users in finding qualified real estate agents. The terms “real estate,” “property,” and “home” are generally used interchangeably herein. System 100 includes a server 110 for providing programming instructions to a plurality of devices 130 over a network 120. In one embodiment, devices 130 are wireless devices, and network 120 includes wireless communication to the device. In general, server 110 may produce programming instructions, files, or data that may be transmitted over network 120 to operate devices 130. In addition, network 120 may also provide access to instructional material which can be retrieved and displayed on device 130.

In general, a user of device 130 may communicate over network 120 to server 110, which includes programming to receive and transmit information with devices 130. FIG. 2 illustrates one embodiment of platform 100 programmed as a system of the present invention.

Server 110 is a computer, a computer system, or network of computers or systems that may include a network interface 111, a memory 113, and a processor 115. Is to be understood that network interface 111, memory 113, and processor 115 are configured such that a program stored in the memory may be executed by the processor to accept input and/or provide output through network interface 111 over network 120 to devices 130.

Devices 130 may be, for example and without limitation, a smartphone, or a desktop or portable computer, tablet computer, or a portable digital assistant, and may include a touchscreen 131 which operates as an input device and an output device, a network interface 132, a memory 133, a processor 134, an audio output 135, position sensors 136, which may include a gyroscope, and a camera 137. Memory 133 contains operating instructions of system 100, which processor 134 interprets to operate touchscreen 131 as an input and output device, provide audio to speakers, headphones, or earbuds using audio output 135, to retrieve information from position sensors 136 to determine the orientation of the device in space, and operate camera 137. While the operation of system 100 is described herein with reference to device 130, the system is not limited to the use of devices having the features of device 130. Thus, it will be understood by those in the field that some of the feature of device 130 may be available on all user devices, such as a camera or position sensors, and that some user devices may have other features, such as other input devices.

Network interface 132 is used by device 130 to communication over a wireless network, such as a cellular telephone or Wi-Fi network, and then to other telephones through a public switched telephone network or to a satellite, or over the Internet. Memory 133 includes programming required to operate device 130 (such as an operating system or virtual machine instructions) and may include portions that store information or programming instructions obtained over network interface 132, or that are input by the user, such as telephone numbers or images from a device camera.

In the following discussion of system 100, devices 130 and/or server 110 are provided with programs stored in memory 133 and 113, respectively, which instruct processor 115 and 134, respectively, to communicate over the network 120, including retrieving data stored in memory 113, and provide output on displays, such as touchscreen 131. More specifically, the following discussion of system 100 references screenshots of the display of touchscreen 131 that are generated using programs stored in memory 133, and which may also be generated from programs stored in memory 113 of server 100 and which are provided over network 120 to device 130.

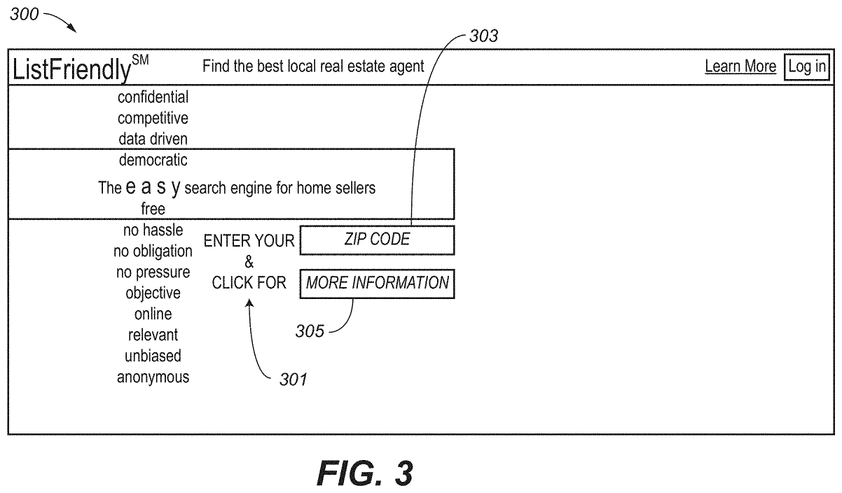

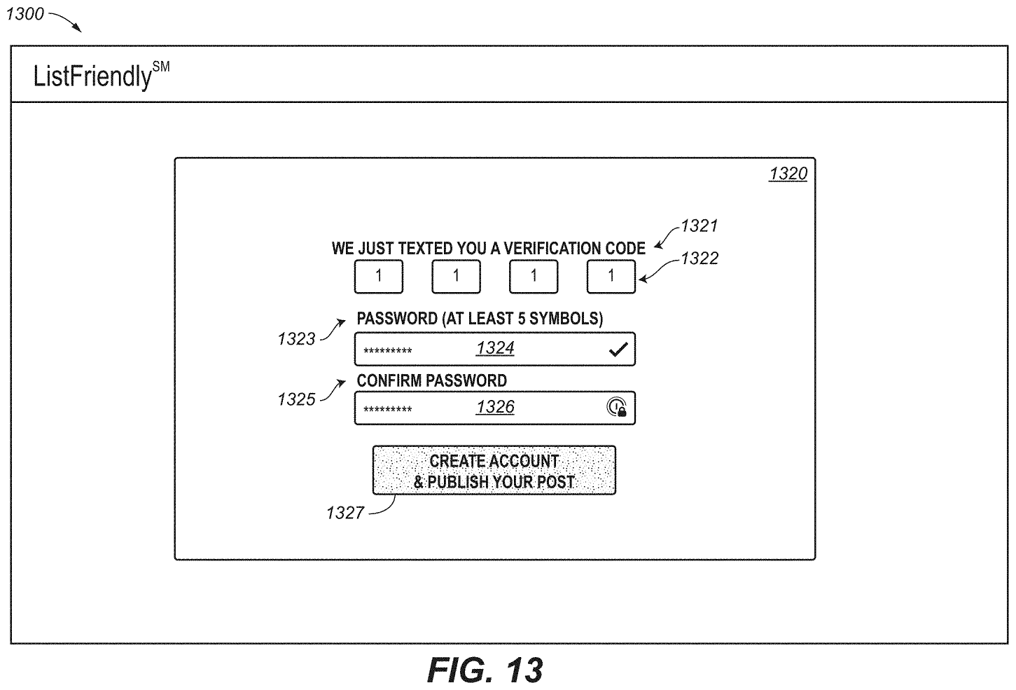

The functioning of system 100 is described herein with reference to FIGS. 3-13 which each show a screenshot of touchscreen 131 for one webpage of a sequence of webpages for one embodiment of system 100. As noted above, the screenshots result from programming stored in memory 113 and/or 133. In various embodiments, one or more of the input provided on device 130 is stored in memory 113 or 133 and may be associated with the user. The input so stored may then be used to report on a specific user's progress and/or may be supplied, in some form, subsequently to device 130 to indicate previous choice or provide default settings.

Optionally, certain embodiments provide an order for the prompts for personal information according to how sensitive the user will be to providing the information. Thus, for example, the personal information may range from not sensitive, such as the user's state of residence, to highly-sensitive, such as the user's phone number or credit card number. In general, the system parses the prompts for personal information with the less sensitive personal information being prompted for in early webpages and the most sensitive personal information in the later webpages.

Thus, for example, in certain embodiments the sensitivity of the requested personal information is ordered from least sensitive to most sensitive for presentation to the user. For the present example, the requested personal information may include, for example and without limitation, the seller's name and contact information and property information. As one example of how the personal information may be ordered by sensitivity, the following is one such ordering: 1) determining the property ZIP code; 2) determining if the seller is a homeowner or a lender; 3) the type of building on the property; 4) the availability of parking and if there are any additional units on the property; 5) the seller's time period for selling the property, the occupancy of the property, determining if the property is a live-work unit, determining if the seller would consider staging the property, if the sale is a short sale, and if anyone smokes at the property; 6) determining if there are pets on the property, if any repairs are needed, and how much the seller believes the property is worth; 7) the number of rooms in the property; 8) request for uploading photographs of the property; and 9) the seller's name and phone number and the property address.

Further, for the present example, system 100 uses the provided personal information to query a real estate database stored in or being accessible to server 110 which contains information similar to that in the local multiple listing service operating in the area where a user's home is located, to obtain information on homes listed and or sold in the previous year. This database includes information about all of the homes listed and sold in the previous year and may include information obtained from a MLS system and/or other third-party websites containing similar information. In an alternative embodiment, system 100 queries a database to which it is networked, such as through the Internet.

In one embodiment, a sequence of eleven screenshots that system 100 provides to touchscreen 131 are illustrated, for example, in FIGS. 3-13, as follows.

FIG. 3 is an embodiment of a screenshot 300 of a first webpage of system 100, which may be a home screen. Screenshot 300 includes information about the value proposition offered by the website—help finding a local real estate agent, a prompt 301 for the ZIP code, a data input region 303, and a “More Information” button 305. A user may input the response in input region 303, and then select the “More Information” button 305, which causes system 100 to store the input to region 303 as the “Property ZIP Code” in the memory of system 100. System 100 also then uses the Property ZIP Code to query a database for home sales information for the Property ZIP Code, which is used to generate a first compelling information in the form of the number of homes sold last year and by how many real estate agents in the Property ZIP Code, as described subsequently.

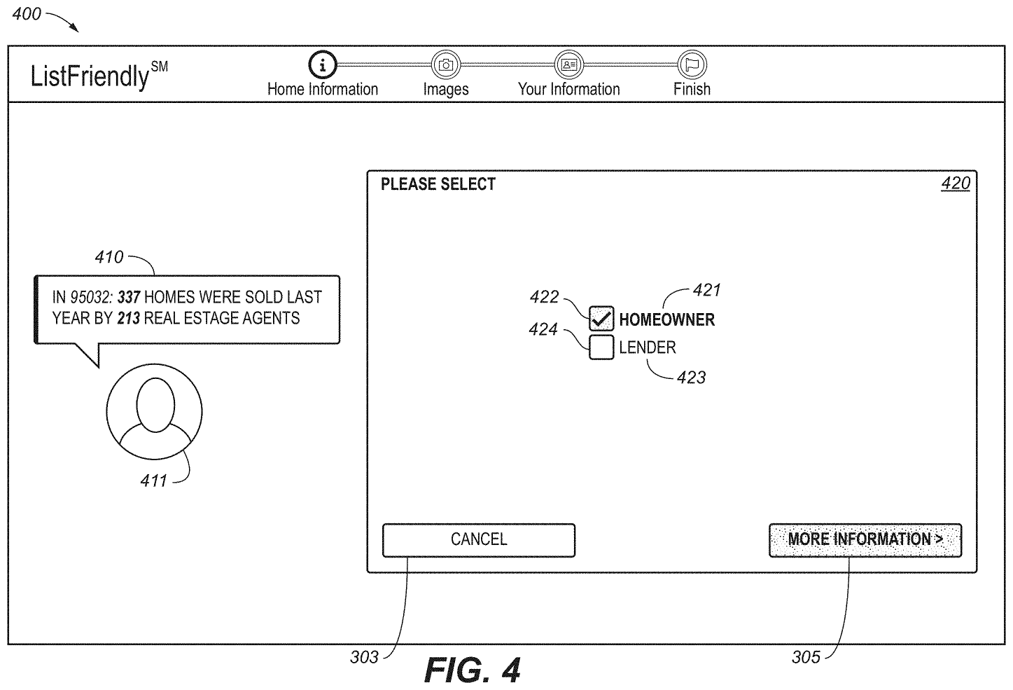

After selecting the “More Information” button 305 from the webpage shown in FIG. 3, system 100 responds by providing a second webpage to device 130, as illustrated for example by screenshot 400 in FIG. 4.

Screenshot 400 includes compelling information 410, a data gathering region 420, a “Cancel” button 403, and “More Information” button 305. Compelling information 410 is the first compelling information, discussed above. Screenshot 400 also includes an image or video 411 that is placed to represent a person providing the compelling information.

Data gathering region 420 includes a first prompt 421 asking if the user is the homeowner and a first input region 422 for entering a response to the first prompt, and a second prompt 423 asking if the user is a lender and a second input region 424 for entering a response to the second prompt. The user may respond to either prompt 422 or prompt 424, but not both.

A user may select the “Cancel” button 403 to exit the sequence of webpages, or may provide input in input regions 422 or 424 and then select the “More Information” button 305, which causes system 100 to store the inputs to input regions 422 or 424 in the memory of system 100. System 100 also then uses the Property ZIP Code to query a database for home sales information and uses the query result to generate a second compelling information in the form of the number of real estate agents currently selling homes of all types, and the number of homes for sale, in the Property ZIP Code.

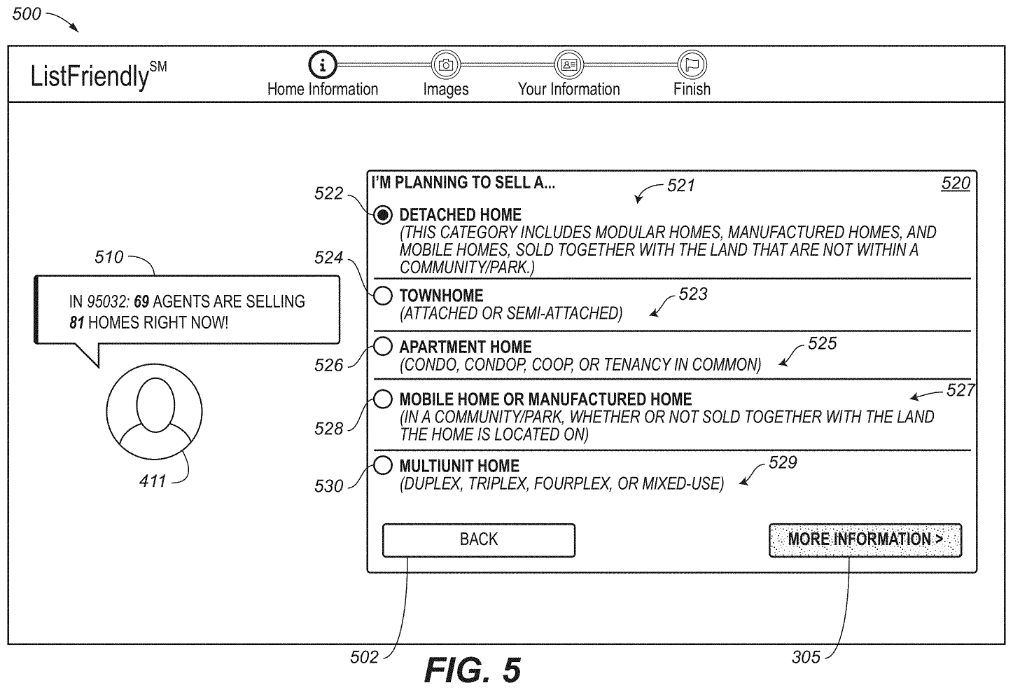

After selecting the “More Information” button 305 from the webpage shown in FIG. 4, system 100 responds by providing a third webpage to device 130, as illustrated for example by screenshot 500 in FIG. 5.

Screenshot 500 includes compelling information 510, an image or video 411 representing the provider of the compelling information, a data gathering region 520, a “Back” button 502 and “More Information” button 305. Compelling information 510 is the second compelling information, discussed above. Data gathering region 520 includes: a first prompt 521 asking if the property type is a detached home and a first data input region 522 for entering a response to the first prompt; a second prompt 523 asking if the property type is a townhome and a second input region 524 for entering a response to the second prompt; a third prompt 525 asking if the property type is an apartment home and a third input region 526 for entering a response to the third prompt; a fourth prompt 527 asking if the property type is a mobile or manufactured home and a fourth input region 528 for entering a response to the fourth prompt; and a fifth prompt 529 asking if the property type is a multiunit home and a fifth input region 530 for entering a response to the fifth prompt. The user may select only one data input; input to region 520 determines the “Property Type.”

A user may select the “Back” button 502 causing system 100 to navigate to the previous webpage, which may be edited, or the user may respond to only one of the prompts and then select the “More Information” button 305, which causes system 100 to store the Property Type as determined from input regions 522, 524, 526, 528, or 530 in the memory of system 100. System 100 also then uses the Property ZIP Code and the Property Type to query a database and obtain home sales information to generate a third compelling information for the Property ZIP Code in the form of the current number of homes of the Property Type currently for sale and the number of agents involved, and the number of homes of the Property Type sold during the previous year and the number of agents involved.

The following screenshots result from a specific response to the input from input region 520, and are thus for a specific Property Type, namely detached homes. System 100 may provide different prompts for different types of property types. While these variations are not described herein, they would be obvious to one skilled in the art.

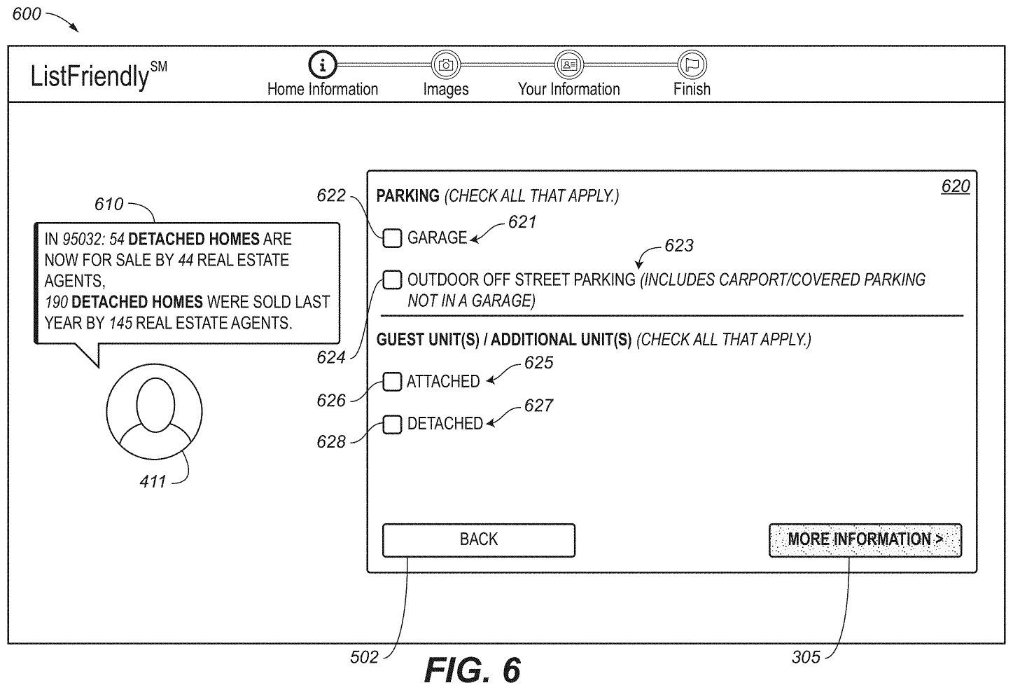

After selecting the “More Information” button 305 from the webpage shown in FIG. 5, system 100 responds by providing a fourth webpage to device 130, as illustrated for example by screenshot 600 in FIG. 6.

Screenshot 600 includes compelling information 610, an image or video 411 representing the provider of the compelling information, a data gathering region 620, “Back” button 502 and “More Information” button 305. Compelling information 610 is the third compelling information, discussed above. Data gathering region 620 includes: a first prompt 621 asking if the property has a garage and a first data input region 622 for responding to the first prompt; a second prompt 623 asking if the property has outdoor off-street parking and a second data input region 624 for responding to the second prompt; a third prompt 625 asking if the property has attached guest or additional units and a data input region 626 for responding to the third prompt; and a fourth prompt 627 asking if the property has detached guest or additional units and a data input region 628 for responding to the fourth prompt.

A user may select the “Back” button 502 causing system 100 to navigate to the previous webpage, which may be edited, or may respond to the prompt responses and then select the “More Information” button 305, which causes system 100 to store the inputs to data input regions 622, 624, 626, and 628 in a memory of system 100. System 100 also then uses the Property ZIP Code and Property Type to query a database for home sales information to generate a fourth compelling information for the Property ZIP Code in the form of the lowest and highest price paid for homes of the Property Type sold in the past year.

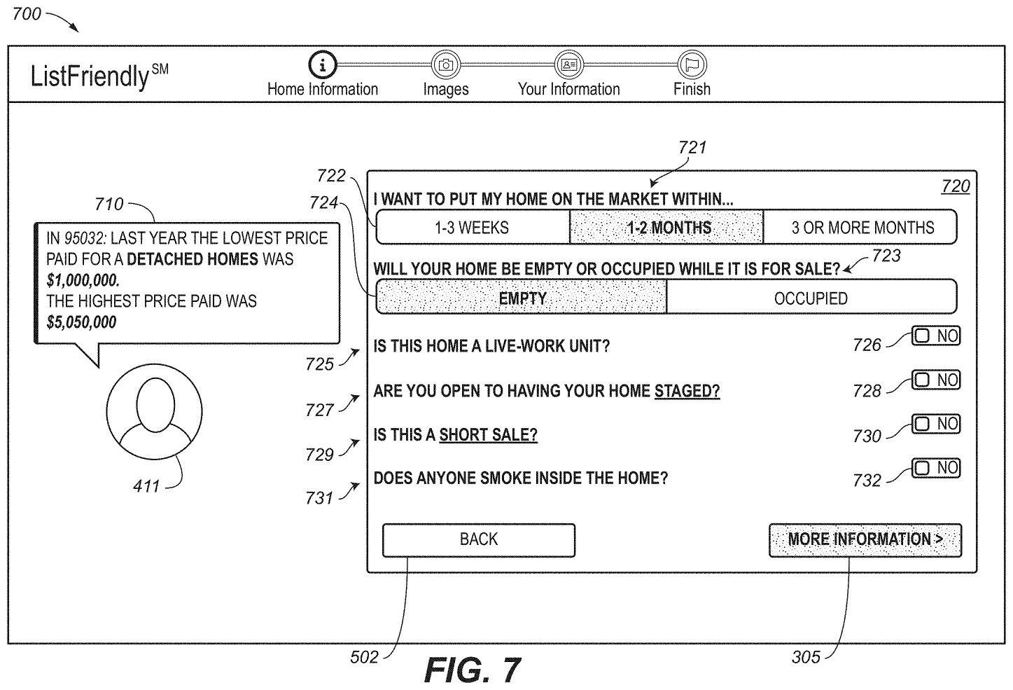

After selecting the “More Information” button 305 from the webpage shown in FIG. 6, system 100 responds by providing a fifth webpage to device 130, as illustrated for example by screenshot 700 in FIG. 7.

Screenshot 700 includes compelling information 710, an image or video 411 representing the provider of the compelling information, a data gathering region 720, “Back” button 502 and “More Information” button 305. Compelling information 710 is the fourth compelling information, discussed above. Data gathering region 720 includes: a first prompt 721 asking when the home will be placed on the market and a first data input region 722 for responding to the first prompt, a second prompt 723 asking if the home will be occupied or empty when it is put up for sale and a second data input region 724 for responding to the second prompt; a third prompt 725 asking if the home is a live-work unit and a third data input region 726 for responding to the third prompt; a fourth prompt 727 asking if the user is open to having the home staged and a fourth data input region 728 for responding to the fourth prompt; a fifth prompt 729 asking if this is a short sale, and a fifth data input region 730 for responding to the fifth prompt; and a sixth prompt 731 asking if anyone smokes inside the home and a sixth data input region 732 for responding to the sixth prompt.

A user may select the “Back” button 502 causing system 100 to navigate to the previous webpage, which may be edited, or may respond to the prompt responses and then select the “More Information” button 305, which causes system 100 to store the inputs to input regions 722, 724, 726, 728, 730, and 732 in memory of system 100. System 100 also then uses the Property ZIP Code and Property Type to query a database for home sales information to generate a fifth compelling information for the Property ZIP Code and Property Type in the form of a the least and the most number of days such properties took to sell during the previous year, as well as the average number of days on the market for such properties.

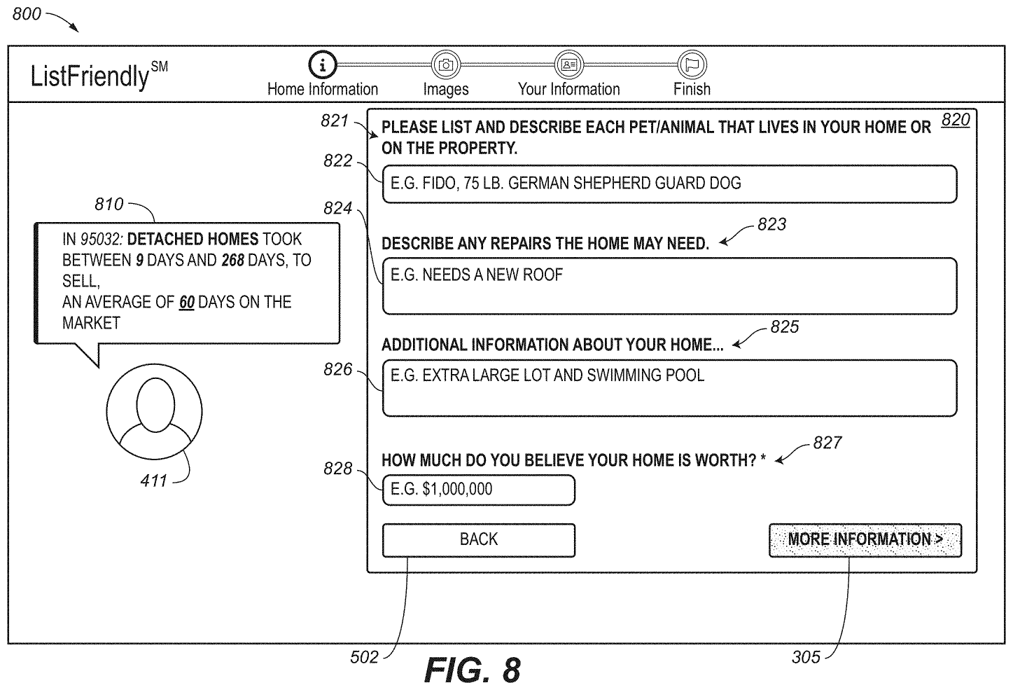

After selecting the “More Information” button 305 from the webpage shown in FIG. 7, system 100 responds by providing a sixth webpage to device 130, as illustrated for example by screenshot 800 in FIG. 8.

Screenshot 800 includes compelling information 810, an image or video 411 representing the provider of the compelling information, a data gathering region 820, “Back” button 502 and “More Information” button 305. Compelling information 810 is the fifth compelling information, discussed above. Data gathering region 820 includes: a first prompt 821 asking for personal information regarding pets or animals on the property and a first data input region 822 for responding to the first prompt, a second prompt 823 asking for a description of any needed repairs to the property and a second data input region 824 for responding to the second prompt; a third prompt 825 asking for additional information about the home and a third data input region 826 for responding to the third prompt; and a fourth prompt 827 asking how much the user believes the home to be worth and a fourth data input region 828 for responding to the fourth prompt and accepting a “Estimated Home Value.”

A user may select the “Back” button 502 causing system 100 to navigate to the previous webpage, which may be edited, or may respond to the prompt responses and then select the “More Information” button 305, which causes system 100 to store the inputs to input regions 822, 824, 826, and 828 in memory of system 100. System 100 also then uses the Property ZIP Code, the Property Type, and Estimated Home Value to query a database for home sales information to generate a sixth compelling information for the Property ZIP code and Property Type in the form of the difference between the asking price and the selling price for homes within 10% of the Estimated Home Value.

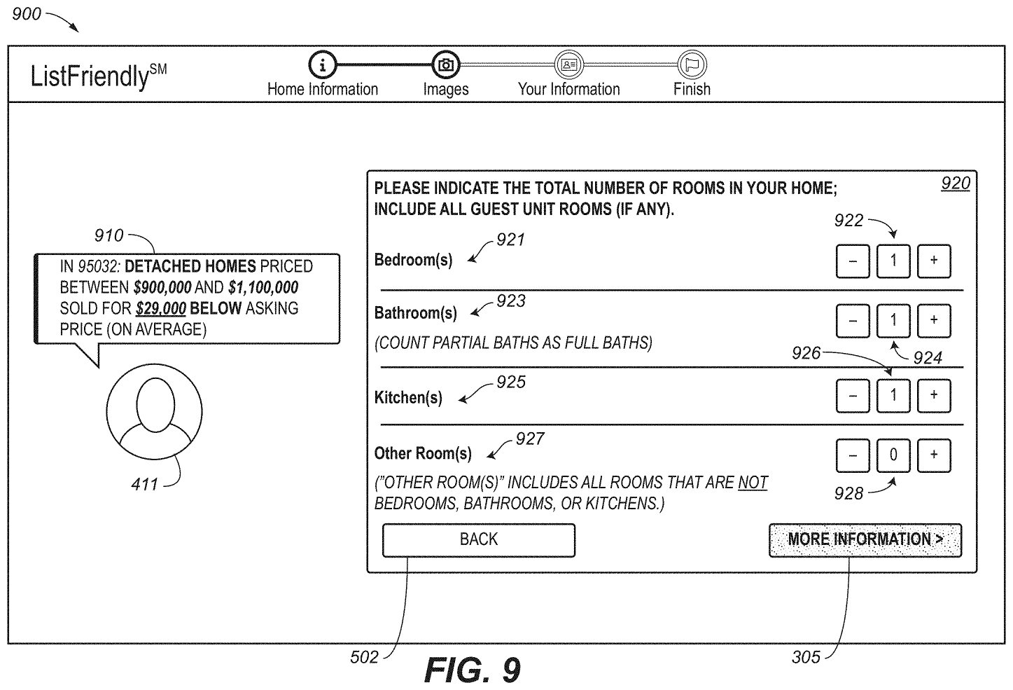

After selecting the “More Information” button 305 from the webpage shown in FIG. 8, system 100 responds by providing a seventh webpage to device 130, as illustrated for example by screenshot 900 in FIG. 9.

Screenshot 900 includes compelling information 910, an image or video 411 representing the provider of the compelling information, a data gathering region 920, “Back” button 502 and “More Information” button 305. Compelling information 910 is the sixth compelling information, discussed above. Data gathering region 920 includes: a first prompt 921 asking for the number of bedrooms in the property and a first data input region 922 for responding to the first prompt with the number of “Bedroom(s)” a second prompt 923 asking for the number of bathrooms in the property and a second data input region 924 for responding to the second prompt with the number of “Bathroom(s);” a third prompt 925 asking for the number of kitchens in the property and a third data input region 926 for responding to the third prompt; and a fourth prompt 927 asking for the number of other rooms in the property and a fourth data input region 928 for responding to the fourth prompt.

A user may select the “Back” button 502 causing system 100 to navigate to the previous webpage, which may be edited, or may respond to the prompt responses and then select the “More Information” button 305, which causes system 100 to store the inputs to input regions 922, 924, 926, and 928 in memory of system 100. System 100 also then uses the Property ZIP Code, Property Type, the Number of Bedrooms, and the Number of Bathrooms to query a database for home sales information to generate a seventh compelling information for the Property ZIP code and Property Type having the Number of Bedrooms and Number of Bathrooms in the form of the number of homes sold in the past year.

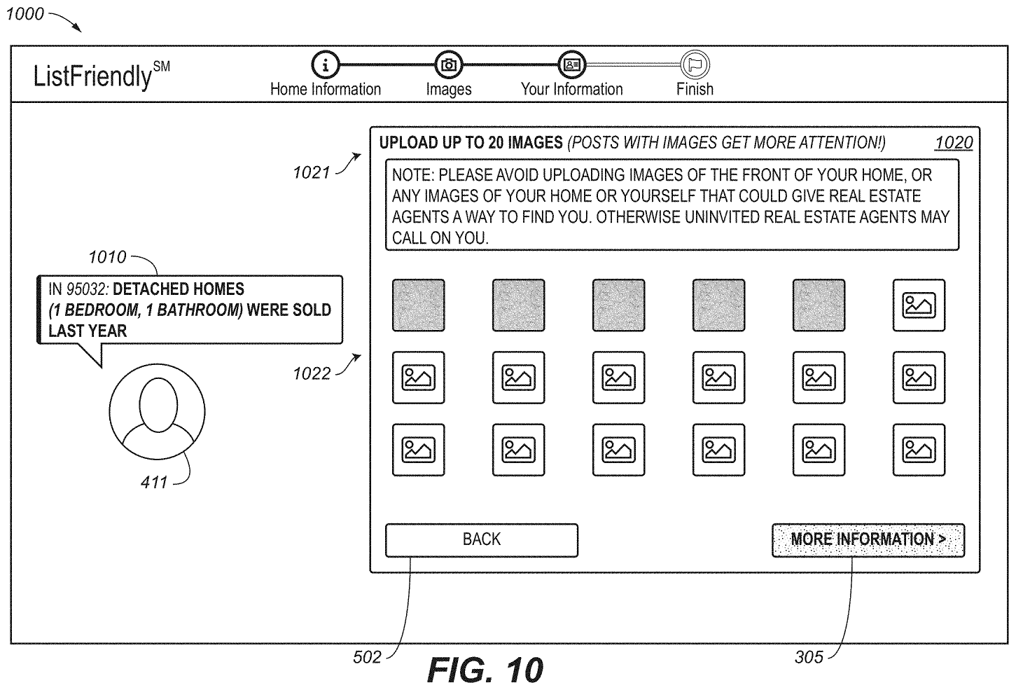

After selecting the “More Information” button 305 from the webpage shown in FIG. 9, system 100 responds by providing an eighth webpage to device 130, as illustrated for example by screenshot 1000 in FIG. 10.

Screenshot 1000 includes compelling information 1010, an image or video 411 representing the provider of the compelling information, a data gathering region 1020, “Back” button 502 and “More Information” button 305. Compelling information 1010 is the seventh compelling information, discussed above. Data gathering region 1020 includes: a prompt 1021 asking for the user to upload photographs of the property and a data input region 1022 for uploading photographs.

A user may select the “Back” button 502 causing system 100 to navigate to the previous webpage, which may be edited, or may respond to the prompt responses and then select the “More Information” button 305, which causes system 100 to store the photographs uploaded in input region 1022 in memory of system 100. System 100 uses the Property ZIP Code, Property Type, Number of Bedrooms, and Number of Bathrooms to query a database for home sales information to generate an eighth compelling information for the Property ZIP code and Property Type with the Number of Bedrooms and Number of Bedrooms, in the form of the number of homes that are currently for sale.

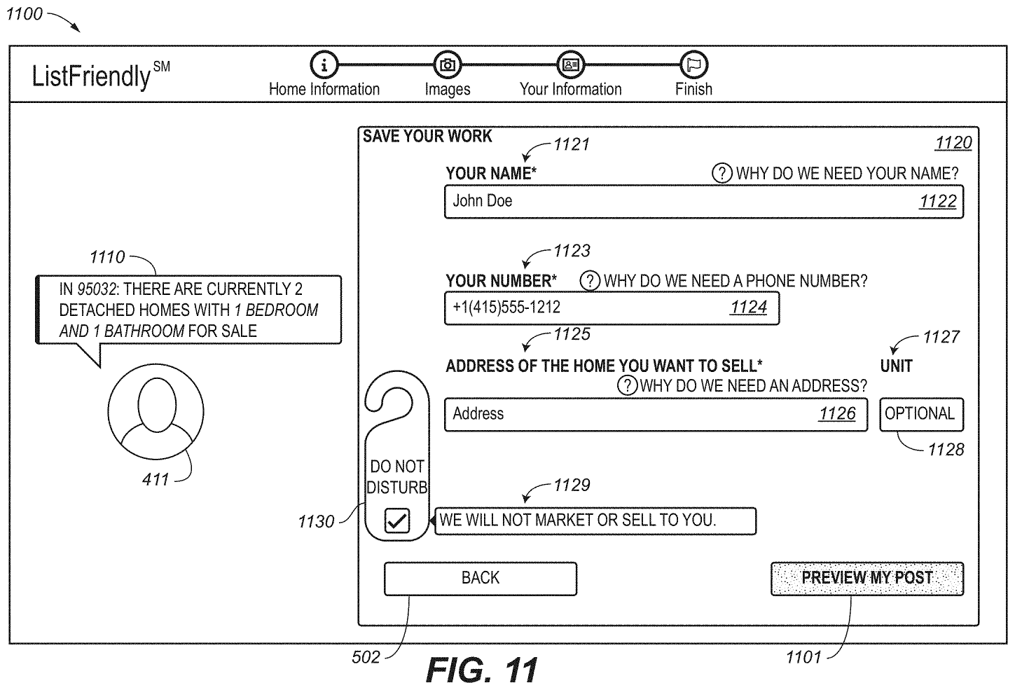

After selecting the “More Information” button 305 from the webpage shown in FIG. 10, system 100 responds by providing a ninth webpage to device 130, as illustrated for example by screenshot 1100 in FIG. 11.

Screenshot 1100 includes compelling information 1110, an image or video 411 representing the provider of the compelling information, a data gathering region 1120, “Back” button 502 and a “Preview My Post” button 1101. Compelling information 1110 is the eighth compelling information, discussed above. Data gathering region 1120 includes: a first prompt 1021 asking for the name of the user and a first data input region 1122 for responding to the first prompt; a second prompt 1123 asking for the phone number of the user and a second data input region 1124 for responding to the second prompt; a third prompt 1125 asking for the address of the property to be sold and a third data input region 1126 for responding to the third prompt; and a fourth prompt 1127 asking for optional unit number of the property to be sold and a fourth data input region 1228 for responding to the fourth prompt.

Data gathering region 1120 also includes an optional fifth prompt 1129 which relates to user privacy. Specifically, fifth prompt 1129 asks if the user wishes to opt-out of receiving marketing or sales communications and a data input region 1130 marked “Do Not Disturb” for responding to the fifth prompt. Fifth prompt 1129 and its response provide the user with the ability to limit the website's use of personal information as an inducement to the user to provide their personal information.

A user may select the “Back” button 502 causing system 100 to navigate to the previous webpage, which may be edited, or may respond to the prompt responses and then select the “Preview My Post” button 1101, which causes system 100 to store the inputs to input regions 1122, 1124, 1126, 1128, and 1130 in memory of system 100. System 100 uses the Property ZIP Code to generate a ninth compelling information for the Property ZIP code in the form of the number of real estate agents registered on the website that operate within the zip code and would provide their services to the user.

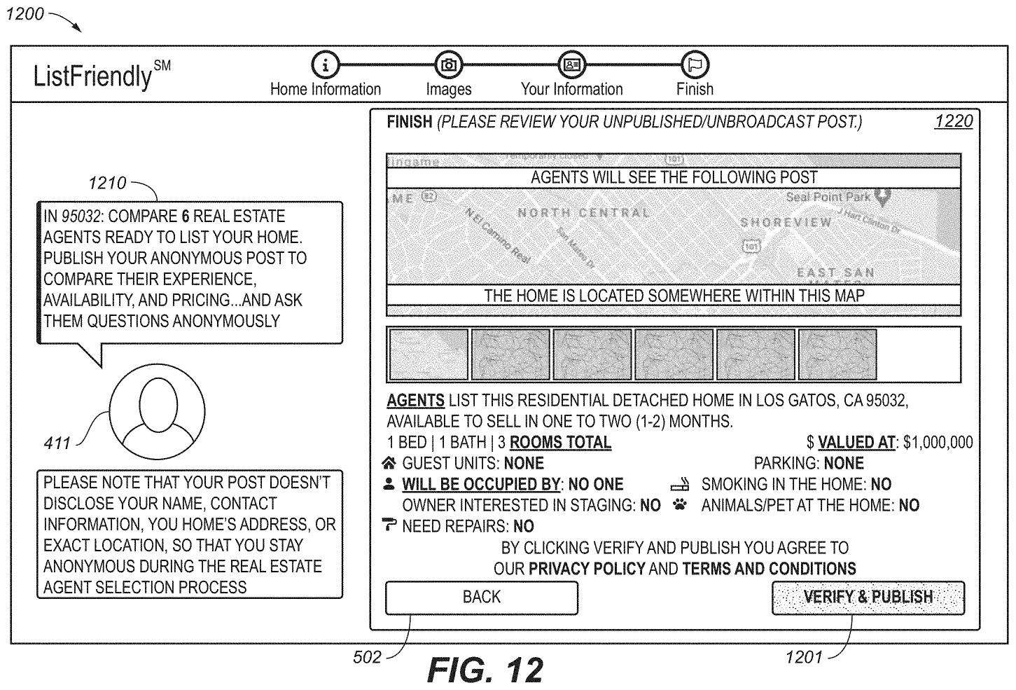

After selecting the “More Information” button 305 from the webpage shown in FIG. 11, system 100 responds by providing a tenth webpage to device 130, as illustrated for example by screenshot 1200 in FIG. 12.

Screenshot 1200 includes compelling information 1210, an image or video 411 representing the provider of the compelling information, a review region 1220, a “Back” button 502, and a “Verify & Publish” button 1201. Review region 1220 includes a summary of the personal information provided, as discussed regarding screenshots 300, 400, 500, 600, 700, 800, 900, 1000, and 1100. Compelling information 1210 is the ninth compelling information, discussed above.

After reviewing the personal information in review region 1220, a user may select the “Back” button 502 causing system 100 to navigate to the previous webpage, which may be edited, or to accept the input and select the “Verify & Publish” button 1201,

After selecting the “Verify & Publish” button 1201 from the webpage shown in FIG. 12, system 100 responds by sending a text message with a verification code to the telephone number provided in data input region 1124 and by providing an eleventh webpage to device 130, as illustrated by screenshot 1300.

At this point, the user has registered with the website and is provided with additional access and information from system 100.

In addition to the compelling information described above, system 100 may also provide static compelling information, such as general information of interest to sellers of real estate.

The system and method described above are in terms of a website related to real estate. These embodiments are not meant to limit the scope of the present invention, which may be used for websites directed to other subject matter including but not limited to, travel, such as making travel arrangements, selling merchandise such as perfume, and arranging for the providing of services such as healthcare, including but not limited to diagnosing illnesses.

As an example of another embodiment, the inventive system and method may be used in a healthcare website directed to diagnosing medical conditions. Thus, for example, the questionnaire may be in the form of a series of questions following a logic tree for diagnosing the cause and treatment of an infant's cough.

The personal information for this example may include an infant's age, and measures or indications of the infant's heartrate, breathing, fever, type of cough, nose, skin, eyes, bodily swelling, and stomach. As the user progresses through to the end of the questionnaire the user is provided with increasingly more probable diagnoses. At the end of the questionnaire, the user is provided with probable diagnosis and instructions for further care.

A first webpage includes the prompt “how old is your infant,” and may for example, permit one of three possible answers: up to 1 month; from 1 to 11 months; and from 12 to 24 months.

A second webpage is used to determine the infant's heartrate. This page may include compelling information in the form of “interesting facts,” which may either be static or dynamic. Examples of an interesting fact includes the static fact that “It is normal for an infant's breathing to vary, sometimes exceeding 60 breaths per minute and sometimes stopping for five to 10 seconds.” Another example of an interesting fact is a dynamic fact based on the infant's age as: “For infants up to one month old, the normal heart rate range is between 70 and 190 beats per minute.”

Another type of compelling information are instructions and/or explanations. An example of this type of compelling information on the heart rate page is instructions on how to measure an infant's heartrate, such as: “Gently press two fingers (don't use your thumb) on the spot until you feel a beat. When you feel the pulse, count the beats for 15 seconds. Multiply the number of beats you counted by four to get the beats per minute.” The heartrate webpage may also prompt for the measured heart rate and provide a region for the use to input the measured heart rate, such as providing the choice between: below 70; 70-190; and above 190.

Another type of compelling information is a matrix showing possible diagnoses. This dynamic matrix provides a table of possible diagnoses and a probability based on previous input. The matrix of possible diagnoses uses as input the responses to the prompts and is programmed with a medical expert's understanding to provide the probabilities that the cough is likely due to one or another of a number of causes. Examples of diagnoses may include, but are not limited to: viral upper respiratory infection; respiratory syncytial virus (RSV); parainfluenza; transient tachypnea; sepsis; bronchiolitis; infant respiratory distress syndrome; metapneumovirus; influenza; pertussis; pneumothorax; pneumonia; asthma; and heart disease.

Thus, while the matrix of possible diagnoses does not provide a definitive diagnosis, it displays the probability (from 0 to 100%) for a variety of possible diagnoses, which changes as the user provides more information. The matrix may take the form of a grid displaying common illnesses on its Y axis—occurring in 95% of all infant patients where a cough is one of the symptoms. The X axis is a set of rows—one for each illness—a continuum measuring the probability of the occurrence of the particular illness from 0 to 100% based on the user's answers to queries about symptoms. For each symptom, the percentage score is increased for every illness (represented on the grid) that may present with the particular symptom indicated by the user.

A third webpage is used to provide information related to the infant's breathing. Static compelling information may include instructions on how to measure an infant's rate of breathing and general information based on the infant's age, such as: “A newborn takes 30 to 60 breaths per minute. This can slow down to 20 when sleeping,” or “At six months babies typically breath 25 to 40 times per minute. It's normal for an infant's breathing to vary, sometimes exceeding 60 breaths per minute and sometimes stopping for five to 10 seconds.”

The third webpage also includes a first query for the infant's breathing rate and permits the user to input breathing rate, and a second query describing different breathing observations, such as: wheezing; pauses while breathing for longer than 10 seconds; noisy harsh breathing; hoarse voice or cry; grunts when exhaling; skin retracts under the rib cage or between the ribs when breathing; whooping sound breathing in; using non breathing muscles to aid breathing; labored breathing; shortness of breath; and panting. Further informational compelling information may be provided for each of these choices to an in depth explanation of how to identify panting, or a recording of what e.g. a panting infant sounds like.

As the information is input to the third webpage, system 100 updates the probabilities in the matrix of possible diagnosis using this information, along with the age and heartrate.

Another type of compelling information that can be provided as the user provides input is a matrix of care options. The matrix of care options uses as input the responses to the prompts and is programmed with a medical expert's understanding to provide an opinion on the possible care options. Depending on the user's selections, the matrix of care options changes to reflect this expert understanding, and may suggest for example, an indication to call your doctor, visit urgent care, proceed to the emergency room, or call an ambulance.

The website could also gather and disseminate other information, such as whether the infant is suffering from more than one illness at the same time, or collect zip code information and/or insurance information which would then be used to make medical care provider recommendations to the user.

One embodiment of each of the methods described herein is in the form of a computer program that executes on a processing system, e.g., a one or more processors that may be part of a network. Thus, as will be appreciated by those skilled in the art, embodiments of the present invention may be embodied as a method, an apparatus such as a special purpose apparatus, an apparatus such as a data processing system, or a carrier medium, e.g., a computer program product. The carrier medium carries one or more computer readable code segments for controlling a processing system to implement a method. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code segments embodied in the medium. Any suitable computer readable medium may be used including a magnetic storage device such as a diskette or a hard disk, or an optical storage device such as a CD-ROM.

It will also be understood that the invention is not limited to any particular implementation or programming technique and that the invention may be implemented using any appropriate techniques for implementing the functionality described herein. The invention is not limited to any particular programming language or operating system.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Thus, while there has been described what is believed to be the preferred embodiments of the invention, those skilled in the art will recognize that other and further modifications may be made thereto without departing from the spirit of the invention, and it is intended to claim all such changes and modifications as fall within the scope of the invention. For example, in some or all of the screenshots, there is no image or video placed to present a person providing the compelling information. Functionality may be added or deleted from the block diagrams and operations may be interchanged among functional blocks. Steps may be added or deleted to methods described within the scope of the present invention.

1. A method of providing an on-line questionnaire for gathering personal information from a user, said method comprising:
providing a plurality of prompts for the user's personal information, where the plurality of prompts have a sequence including a last prompt;
accepting a response from the user for each prompt of the plurality of prompts;
determining one or more items of compelling information from one or more accepted responses; and
providing the one or more items of determined compelling information to the user, where at least one of the one or more items of determined compelling information is provided to the user before the step of accepting the response to the last prompt,
such that the user is presented with compelling information while responding to the on-line questionnaire.

2. The method of claim 1, where the providing of the plurality of prompts includes providing one or more webpages each including:
one or more prompts of the plurality of prompts; and
a region for accepting responses to the prompts on the webpage.

3. The method of claim 2, where the provided determined compelling information provides the compelling information on a webpage.

4. The method of claim 1 further comprising:
ranking at least two prompts of the plurality of prompts according to the sensitivity to the user of the information prompted, where the ranking ranges from least sensitive to most sensitive; and
sequentially providing the plurality of prompts according to the ranking of the prompts, where the most sensitive prompts are presented last.

5. The method of claim 1, where the determining compelling information includes determining compelling information from a database lookup from the one or more accepted responses.

6. The method of claim 1, where one prompt of the plurality of prompts requests if the user wishes to opt-out of marketing or sales communications, and where, if the user responds in the affirmative, the website's operator will not provide marketing or sales communications to the user.

7. The method of claim 1, where the on-line questionnaire is related to selling the user's real estate, where the plurality of prompts includes prompts for information related to the user's real estate, and where the compelling information includes real estate sales information.

8. The method of claim 7, where the compelling information is obtained from a database of real estate sales data.

9. The method of claim 1, where the on-line questionnaire is related to diagnosing a medical condition, making travel arrangements, selling merchandise, or arranging for the providing of services.
where the plurality of prompts includes prompts for information for diagnosing a medical condition,

10. The method of claim 1, further comprising, for one or more of the one or more items of determined compelling information, providing an image or video representing a person providing the compelling information.

11. A system for providing an on-line questionnaire to gather personal information from a user, said system comprising a server programmed to:
provide a plurality of prompts for the user's personal information, where the plurality of prompts have a sequence including a last prompt;
accept a response from the user for each prompt of the plurality of prompts;
determine one or more items of compelling information from one or more accepted responses; and
provide the one or more items of determined compelling information to the user, where at least one of the one or more items of determined compelling information is provided to the user before the response to the last prompt is accepted,
such that the user is presented with compelling information while responding to the on-line questionnaire.

12. The system of claim 11, where the server is further programmed to provide the plurality of prompts on one or more webpages each including:
one or more prompts of the plurality of prompts; and
a region for accepting responses to the prompts on the webpage.

13. The system of claim 12, where the server is further programmed to provide determined compelling information on a webpage.

14. The system of claim 11, where the server is further programmed to:
accept a ranking of at least two prompts of the plurality of prompts according to the user's sensitivity to the information prompted, where the ranking ranges from least sensitive to most sensitive; and
sequentially provide the plurality of prompts according to the ranking of the prompts, where the most sensitive prompts are presented last.

15. The system of claim 11, where the server is further programmed to determine compelling information from a database lookup from the one or more accepted responses.

16. The system of claim 11, where one prompt of the plurality of prompts requests if the user wishes to opt-out of marketing or sales communications, and where, if the user responds in to opt-out of marketing or sales communication, alert an operator of the system.

17. The system of claim 11, where the on-line questionnaire is related to the selling of the user's real estate, where the plurality of prompts includes prompts for personal information related to the user's real estate, and where the compelling information includes real estate sales information.

18. The system of claim 17, where the compelling information is obtained from a database of real estate sales data.

19. The system of claim 11, where the on-line questionnaire is related to diagnosing a medical condition, making travel arrangements, selling merchandise, or arranging for the providing of services.

20. The system of claim 11, where the server is further programmed, for one or more of the one or more items of determined compelling information, to provide an image or video representing a person providing the compelling information.
